(19) United States
(12) Patent Application Publication
(10) Pub. No.: US 2025/0388949 A1
(43) Pub. Date: Dec. 25, 2025

(54) FIBRILLAR HYDROGEL AND USES THEREOF

(71) Applicant: ALEPH FARMS LTD., Rehovot (IL)

(72) Inventors: Neta Lavon, Ness Ziona (IL); Yair Glick, Petah Tikva (IL); Keren Tazat Fireberg, Ramat Gan (IL)

(21) Appl. No.: 18/865,190

(22) PCT Filed: May 28, 2023
(86) PCT No.: PCT/IL2023/050545

§ 371 Date: Nov. 12, 2024

Related U.S. Application Data
(60) Provisional application No. 63/346,867, filed on May 29, 2022.

(51) Int. Cl. C12P 21/00; C12N 5/0735; C12P 21/02

(52) U.S. Cl. CPC C12P 21/005; C12N 5/0606; C12P 21/02; C12N 2501/999

(57) ABSTRACT

The present invention relates to a fibrillar hydrogel comprising an aqueous solution and substantially aligned fibrils composed of collagen and at least one proteoglycan, having viscoelastic characteristics enabling its use in a wide range of industries, including but not limited to the food industry, biomaterial industry, pharmaceutic industry, and cosmetics, as well as for research purposes.

### CROSS-REFERENCE TO RELATED APPLICATIONS

This application is the U.S. National Stage of International Patent Application No. PCT/IL2023/050545, filed on May 28, 2023, which claims the benefit of and priority to U.S. Provisional Application No. 63/346,867, filed on May 29, 2022, the contents of each of which are hereby incorporated by reference in their entireties.

### INCORPORATION-BY-REFERENCE OF MATERIAL ELECTRONICALLY FILED

The official copy of the sequence listing is submitted electronically in ST.26 XML format having the file name “ALPH014 SL.xml” created on Feb. 6, 2025, and having a size of 5.47 bytes, and is filed concurrently with the specification. The Sequence Listing ST.26 XML file is part of the specification and is herein incorporated by reference in its entirety.

### FILED OF THE INVENTION

The present invention relates to a fibrillar hydrogel comprising an aqueous solution and substantially aligned fibrils composed of collagen and at least one proteoglycan, having viscoelastic characteristics enabling its use in a wide range of industries, including but not limited to the food industry, biomaterial industry, pharmaceutic industry, and cosmetics, as well as for research purposes.

### BACKGROUND OF THE INVENTION

The extracellular matrix (ECM) is an aggregation of macromolecules, arranged in a three-dimensional network designed to give structural and functional support to the cells enmeshed within it. The ECM is comprised of various types of macromolecules, including but not limited to collagens, elastin, fibronectin, laminins, glycoproteins, proteoglycans and glycosaminoglycans (GAGs). The exact composition of the ECM varies from tissue to tissue, and adequately, mimicking this highly critical supportive environment is crucial for areas such as tissue engineering and ex-vivo cell culturing. There is an increasing success in producing man-made ECM-like hydrogels, in a variety of methods, albeit with inconsistent characteristics of the produced hydrogel. Challenges to creating a hydrogel that closely resembles the real ECM present in live tissues include batch-to-batch compositional variations, inability to precisely recreate the structural integrity of a natural ECM and reliance on chemical protocols that render the hydrogel biologically incompatible.

Fibrillar hydrogels are a class of man-made hydrogels, which successfully recreate the filamentous architecture of in-vivo ECMs, coupled with the compositional precision awarded by the controlled nature of the synthetic process. Structural integrity alone, however, is not sufficient in regards to creating a functional ECM-mimetic hydrogel. Correct macromolecular composition, which in turn affects the biophysical properties, is highly important for a fibrillar hydrogel to be functional and biologically relevant.

Collagen is a major ECM component of certain animal species, including mammals. The primary role of collagen is to provide a scaffold to support tissues, although a number of other functions have been elucidated for collagen, including roles in cell attachment, cell migration, filtration and morphogenesis. Skin, or animal hide, contains significant amounts of collagen.

“Collagen” is a name referring to a super family of proteins characterized by a triple-helix domain which composes more than 95% of the molecule. The domain consists of three alpha chains, each containing approximately 1,000 amino acids, wrapped in a rope-like fashion to form a tight, triple helix structure. Each alpha chain is composed of a repeating triplet of amino acids,-(Gly-X-Y) n-, with glycine being about one-third of the amino acid residues in the collagen. X is often proline and Y is often hydroxyproline, though there may be up to 400 possible Gly-X-Y triplets. The hydroxylation of proline residues is crucial for helical integrity, and defects in this process result in widespread defects throughout connective tissues. Different animals may produce different amino acid compositions of the collagen, which may result in different collagen properties. The collagen structure consists of three intertwined peptide chains of differing lengths, forming the collagen triple helices.

During production of extracellular matrix by skin fibroblast cells, triple helix monomers are synthesized and the monomers may self-assemble into a fibrous form. These triple helices are held together by electrostatic interactions including salt bridging, hydrogen bonding, Van der Waals interactions, dipole-dipole forces, polarization forces, hydrophobic interactions, and/or covalent bonding. The triple helices can be bound together in bundles called fibrils, and fibrils can further assemble to create fibers and fiber bundles. Fibrils have a characteristic banded appearance due to the alternated overlap of collagen monomers. Fibrils and fibers typically branch and interact with each other throughout a layer of skin or hide. Variations of the organization or crosslinking of fibrils and fibers may provide strength to the material.

Currently, there are 28 known distinct collagen types. Collagen types are numbered by Roman numerals, and the chains found in each collagen type are identified by Arabic numerals. Detailed descriptions of structure and biological functions of the various types of naturally occurring collagens are available in the art (e.g., Ayad et al. 1998. The Extracellular Matrix Facts Book, Academic Press, San Diego, CA).

Collagen Type I is the most prevalent form of collagen in mammalian species and is ubiquitously distributed throughout the body in skin, bone, muscle, tendon, and lung. It is the major structural macromolecule present in the extracellular matrix of multicellular organisms and comprises approximately 20% of total protein mass. Type I collagen is a heterotrimeric molecule comprising two α1(I) chains and one α2(I) chain. Other collagen types are less abundant than type I collagen, and exhibit different distribution patterns. For example, type II collagen is the predominant collagen in cartilage and vitreous humor. Type III collagen is found as a major structural component in hollow organs such as large blood vessels, uterus, and bowel. It is also found in many other tissues together with type I collagen.

Collagen has been successfully isolated from various regions of the mammalian body in addition to the animal skin or hide. In more recent years, collagen has been harvested from bacteria and yeast using recombinant techniques.

International (PCT) Application Publication No. WO 2022/043994 to the Applicant of the present invention provides systems and methods for the production of cell-free animal collagen and/or a medium comprising this collagen. The systems and methods of the invention include a cell culture continuously producing soluble collagen that is secreted into the culture medium, thus enabling easy collection of the soluble collagen. The soluble collagen can thereafter be processed to form collagen fibrils and fibers, and the collagen products can be used in medicine or cosmetics, in the cell cultured food industry or can be further bio-fabricated to form leather-like textile.

International (PCT) Application Publication No. WO 2023/017509 to the Applicant of the present invention provides compositions and methods for producing lineage-committed progenitor cells from pluripotent stem cells and cells differentiated therefrom, including, inter alia, ECM-producing cells, including collagen-producing cells.

The beneficial characteristics of collagen has led to its use in tissue engineering and in a variety of biomedical applications, and a vast effort has been made to produce high-quality collagen. Medical and cosmetic applications of collagen include, for example, skin fillers, wound dressing, and guided tissue regeneration.

International (PCT) Application Publication No. WO 2018/046920, for example, discloses a method for producing jellyfish collagen hydrogels and kits for producing the same. The jellyfish collagen hydrogels can be used in the manufacture of 3D cell culture scaffolds and in the manufacture of medical devices.

Collagen scaffolds have been widely used in tissue engineering as they offer low immunogenicity, a porous structure, good permeability, biocompatibility, and biodegradability. These scaffold structures serve as templates with specific mechanical and biological properties similar to native extracellular matrix (ECM).

Collagen is also a primary component in many cosmetic formulations since it is a natural humectant and moisturizer. Hydrolyzed Collagen is used primarily in hair preparations and skin care products, but can also be found in makeup, shampoos, and bath products. Hydrolyzed Collagen may also be used in hair dyes.

Leather is used in a vast variety of applications, including furniture upholstery, clothing, shoes, luggage, handbags, and accessories, as well as in automotive applications. The global trade value of leather is estimated at US $100 billion per year and there is a continuing and increasing demand for leather products. However, use of natural leather from slaughtered animals has encountered public rejection due to increased awareness to animal welfare, use of chemicals hazardous to the environment and health issues. New ways to meet the demand for leather materials amenable to large scale production and exhibiting at least equal or superior properties compared to natural leather are thus required (for example, International (PCT) Application Publication Nos. WO 2017/003999, and WO 2017/142887; U.S. Application Publication No. 2019/0203000).

U.S. Pat. No. 9,644,177 discloses films formed from type I collagen serving as a model for the basal lamina, possibly used to form complex three-dimensional structures. The film described therein is formed from Type I collagen and one or more additional ECM proteins, such as additional types of collagen, laminin, vitronectin, fibronectin, and chondroitin sulfate proteoglycans and comprises a bilaminar surface.

There is an unmet need for an adequately mimetic hydrogel to be used as a synthetic ECM, possessing the qualities of an in-vivo ECM, including structural integrity, compositional precision, correct biophysical properties and biological compatibility. A fibrillar hydrogel of this nature is essential for myriad industries, including but not limited to tissue engineering and cell culturing, the cosmetic and food industries, leather-mimetic industry, and more.

### SUMMARY OF THE INVENTION

The present invention provides fibrillar hydrogels comprised of an aqueous solution, typically a buffer or water, and a plurality of substantially aligned fibrils composed of animal collagen and at least one type of proteoglycan. In exemplary embodiments, the fibrils and/or the hydrogel further comprise additional extracellular matrix (ECM) proteins, and the pH of the hydrogel aqueous solution is non-acidic. In certain embodiments, the animal is a mammal, including human and non-human mammals. According to certain exemplary embodiments, the animal is a non-human animal. Advantageously, according to certain embodiments, the fibrillar hydrogel of the present invention is substantially free of pyrogens. According to certain exemplary embodiments, the fibrillar hydrogel of the present invention is substantially free of endotoxins.

The present invention is based in part on the unexpected discovery that collagen and proteoglycan(s) secreted from bovine embryonic fibroblast cells and/or from collagen-producing cells differentiated from bovine-derived pluripotent stem cells, can form fibrils that advantageously maintain their fibrillar structure in non-acidic aqueous solution having a pH of at least about 6.00, forming a stable fibrillar hydrogel.

Additional advantage of the fibrillar hydrogel of the present invention is that the fibrils are aligned essentially parallel to each other forming an ordered, multiaxial alignment within the hydrogel, contributing to the viscoelastic characteristics of the hydrogel and to the viscoelasticity of products produced thereof. As yet a further advantage, the fibrillar hydrogel of the invention has a filamentous structure similar to the native ECM filamentous architecture. This advantageously makes the fibrillar hydrogel of the present invention suitable for use as raw material in a variety of industries, including the pharma industry, consumer goods (e.g., non-animal leather), food industry, cosmetics, and more, without requiring additional priming procedures. In a variety of applications, the fibrillar hydrogel may be used directly without a need for significant priming.

According to certain aspects, the present invention provides a fibrillar hydrogel comprising an aqueous solution and a plurality of fibrils, the fibrils comprising collagen and at least one type of proteoglycan, wherein the pH of the hydrogel aqueous solution is at least about 6.00 and wherein at least part of the fibrils are in ordered alignment within said hydrogel.

According to some embodiments, the order alignment is a biaxial alignment. According to certain embodiments, the order alignment is a multiaxial alignment.

According to some embodiments, the fibrils are aligned parallelly.

According to some embodiments, at least about 30%, 35%, 40%, 45%, 50%, 55%, 60%, 65%, 70%, 75%, 80%, 85%, 90% or 95% of the fibrils are in ordered alignment within said hydrogel. Each possibility represents a separate embodiment of the present invention.

According to some embodiments, the fibrils are in ordered alignment at segments of fibril length of from about 100 nm to about 10 mm.

According to certain embodiments, the collagen molecules comprise at least 40% hydroxylated proline and lysine residues out of the total number of proline and lysine residues of said collagen molecules.

According to certain embodiments, the aqueous solution is selected from the group consisting of water, buffer solution and a cell culture medium. Each possibility represents a separate embodiment of the present invention.

According to certain exemplary embodiments, the aqueous solution is water.

According to further exemplary embodiments, the aqueous solution is a buffer solution capable of maintaining a pH of 6.0 and above. According to some embodiments, the buffer solution is capable of maintaining a pH in the range of from about 6.0 to about 8.0.

According to certain embodiments, the aqueous solution content of the fibrillar hydrogel comprises from about 30% to 99%, 35% to 99%, 40% to 99%, 45% to 99%, 45% to 99%, 50% to 99%, 55% to 99%, 60% to 99%, 65% to 99%, 70% to 99%, 75% to 99%, 80% to 99%, 85% to 99%, 90% to 99%, or 95% to 99% w/w out of the total wet weight of the hydrogel. Each possibility represents a separate embodiment of the present invention.

According to certain embodiments, the aqueous solution content of the fibrillar hydrogel comprises from about 90% to about 99%. According to these embodiments, the fibrillar hydrogel comprises a dry weight content of from about 1% to about 10% of the total weight of the hydrogel. Each possibility represents a separate embodiment of the present invention.

According to certain embodiments, the aqueous solution content of the fibrillar hydrogel comprises from about 95% to about 99%. According to these embodiments, the fibrillar hydrogel comprises dry weight content of from about 1% to about 5% of the total wet weight of the hydrogel. Each possibility represents a separate embodiment of the present invention.

According to some embodiments, collagen type I is the predominant type of the collagen comprised in the fibril. According to certain embodiment, collagen type I forms about at least 60% w/w based on dry weight out of the total collagen of the hydrogel. According to certain embodiments, the fibril comprises at least one additional collagen selected from the group consisting of: Collagen III, collagen VI, collagen XII, collagen IV, collagen V, collagen XI, collagen XIV, collagen XVIII, and any combination thereof. Each possibility represents a separate embodiment of the present invention.

According to certain embodiments, the at least one additional collagen is selected from the group consisting of Collagen III, collagen VI, collagen XII and any combination thereof. Each possibility represents a separate embodiment of the present invention.

According to certain embodiments, at least 40%, at least 41%, at least 42%, at least 43%, at least 44%, at least 45%, at least 46%, at least 47%, at least 48%, at least 49%, or at least 50% or more of the total number of the collagen I proline and lysine residues are hydroxylated. Each possibility represents a separate embodiment of the present invention.

According to certain embodiments, the collagen I molecules comprise at least 25% hydroxylated lysine residues out of the total number of lysine residues on the molecule. According to certain embodiments, the percentage of the hydroxylated lysine residues ranges from about 25% to about 35% out of the total lysine residues of collagen I. Each possibility represents a separate embodiment of the present invention.

According to certain embodiments, the collagen I molecules comprise at least 40% of hydroxylated proline residues out of the total number of proline residues on the molecule. According to certain embodiments, the percentage of the hydroxylated proline residues ranges from about 40% to about 60% out of the total proline residues of the collagen I molecule. Each possibility represents a separate embodiment of the present invention.

According to certain embodiments, the at least one proteoglycan is selected from the group consisting of decorin, biglycan, lumican, prolargin, asporin, fibromodulin, versican and any combination thereof. Each possibility represents a separate embodiment of the present invention.

According to some embodiments, the proteoglycan core protein is associated with at least one glycosaminoglycan (GAG) selected from chondroitin sulfate, dermatan sulfate, heparin, heparan sulfate and keratan sulfate. Each possibility represents a separate embodiment of the present invention.

According to some embodiments, the yield stress of the fibrillar hydrogel comprising an aqueous solution content of from about 90% to about 99% at a temperature in a range of from about 22° C. to about 26° C. is from about 1 Pa to about 50 Pa, or from about 1 Pa to about 20 Pa, or from about 5 Pa to about 20 Pa. Each possibility represents a separate embodiment of the invention.

According to certain exemplary embodiments, the yield stress of the fibrillar hydrogel comprising an aqueous solution content of about 90% to about 99% at a temperature of 25° C. is from about 2 Pa to about 20.

According to some embodiments, the phase angle (°) of the fibrillar hydrogel comprising from about 90% to about 99% an aqueous solution content at temperatures of from about 22° C. to about 26° C. is in a range of from about 1° to about 50°, or from about 5° to about 20°. Each possibility represents a separate embodiment of the invention.

According to some embodiments, the phase angle of the fibrillar hydrogel comprising from about 90% to about 99% an aqueous solution content at temperatures of from about 22° C. to about 26° C. is from about 12° to about 20°. Each possibility represents a separate embodiment of the invention.

According to some embodiments, the total amount of collagen is from about 5% to about 95% w/w on a dry weight basis out of the total dry weight of the fibrillar hydrogel. According to certain embodiments, the total amount of collagen is from about 20% to about 90% w/w on a dry basis out of the total dry weight of the fibrillar hydrogel. According to certain exemplary embodiments, the total amount of collagen is from about 70% to about 80% on a dry basis out of the total dry weight of the fibrillar hydrogel.

According to some embodiments, the fibrillar hydrogel further comprises hyaluronic acid.

According to certain embodiments, the fibrillar hydrogel further comprises at least one glycoprotein selected from the group consisting of fibronectin, serpin H1, galectin-1, periostin, laminin, peroxidasin, Elastin Microfibril Interface Located Protein (EMILIN), fibrillin, tenascin C, Secreted protein acidic and rich in cysteine (SPARC), and any combination thereof. Each possibility represents a separate embodiment of the present invention.

According to certain embodiments, the fibrillar hydrogel further comprises at least one protein of the cytoskeleton filaments. According to certain exemplary embodiments, the protein is selected from the group consisting of: actin, vimentin, Myosin, filamin, tropomyosin, transgelin, tubulin and any combination thereof. Each possibility represents a separate embodiment of the present invention.

According to certain embodiments, the fibrillar hydrogel further comprises at least one cell membranal protein. According to certain exemplary embodiments the cell membranes protein is annexin.

According to certain embodiments, the fibrillar hydrogel is produced by animal cells cultured in a culture medium under conditions enabling collagen synthesis.

According to some embodiments, the conditions enabling collagen synthesis comprises the steps of (i) growing said animal cells under conditions enabling at least one of cell proliferation, cell differentiation, cell maturation, and any combinations thereof; (ii) adding to the culture medium at least one agent inducing collagen synthesis.

According to certain embodiments, the agent inducing collagen synthesis is selected from the group consisting of iron, insulin, ascorbic acid or a salt thereof, and a combination of same. Each possibility represents a separate embodiment of the present invention.

According to certain exemplary embodiments, the agent inducing collagen synthesis is ascorbic acid or a salt thereof.

The cells producing collagen can be obtained from humans as well as from any non-human animal having collagen-producing cells.

According to certain embodiments, the collagen-producing animal is selected from the group consisting of mammals, poultry, aquatic animals, invertebrate and reptiles. Each possibility represents a separate embodiment of the present invention.

According to certain embodiments, the collagen producing cells are human cells. According to other embodiments, the collagen producing cells are of non-human animal.

According to some embodiments, the non-human animal is known to be used for its skin or hide, including, but not limited to bovine, sheep, horse, crocodile and the like.

According to some embodiments, the collagen-producing non-human mammal is an ungulate.

According to certain embodiments, the ungulate is selected from the group consisting of a bovine, a buffalo, an ovine, an equine, a pig, a giraffe, a camel, a deer, a hippopotamus, and a rhinoceros. Each possibility represents a separate embodiment of the present invention.

According to certain exemplary embodiments, the ungulae is a bovine.

According to further certain exemplary embodiments, the reptile is of the Crocodylidae family.

According to certain embodiments, the cells are stromal cells. According to certain exemplary embodiments, the stromal cells are fibroblast cells.

According to certain additional or alternative embodiments, the cells are collagen-producing cells differentiated from pluripotent stem cells.

The fibrillar hydrogels of the present invention may be used in a variety of uses. According to certain embodiments, the fibrillar hydrogel is used to form biomaterials (for the textile industry as well as for medical purposes), food, nutraceuticals (including dietary supplements and food additives), cosmetic or medical products. Each possibility represents a separate embodiment of the invention.

According to certain exemplary embodiments, the fibrillar hydrogel of the invention is used in the production of textiles, including leather-like textiles. The natural, cell-free fibrillar hydrogel of the invention is suitable for use in a variety of textile-production methods, as are currently known and as will be known in the art.

According to further exemplary embodiments, the fibrillar hydrogel of the invention is used in the production of food products, particularly cultured meat products.

According to further exemplary embodiments, the fibrillar hydrogel of the present invention is used in the production of medical materials and devices.

According to yet additional exemplary embodiments, the fibrillar hydrogel of the invention is used in the production of cosmetic products.

According to additional certain aspects, the present invention provides a textile comprising at least 10% of the fibrillar hydrogel of the invention. According to certain embodiments, the textile has skin/hide/leather-like characteristics. According to certain embodiments, the textile comprises from about 20% to about 60% of the fibrillar hydrogel. Each possibility represents a separate embodiment of the invention.

According to yet additional certain aspects, the present invention provides a food product comprising at least 0.5% of the fibrillar hydrogel of the invention. According to certain embodiments, the food product comprises from about 1% to about 5% of the fibrillar hydrogel. Each possibility represents a separate embodiment of the invention. According to certain embodiments, the food product is a cell cultured meat product.

According to further certain aspects, the present invention provides a medical material comprising at least 0.1% of the fibrillar hydrogel of the invention. According to certain embodiments, the medical material comprises from about 0.1% to about 1% of the fibrillar hydrogel. Each possibility represents a separate embodiment of the invention.

According to yet further certain aspects, the present invention provides a cosmetic product comprising at least 0.01% of the fibrillar hydrogel of the invention. According to certain embodiments, the cosmetic comprises from about 0.1% to about 1% of the fibrillar hydrogel. Each possibility represents a separate embodiment of the invention.

According to further certain aspects, the present invention provides a nutraceutical product comprising at least 1% of the fibrillar hydrogel of the invention. According to certain embodiments, the nutraceutical product comprises from about 1% to about 20% of the fibrillar hydrogel. Each possibility represents a separate embodiment of the invention.

It is to be understood that any combination of each of the aspects and the embodiments disclosed herein is explicitly encompassed within the disclosure of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 shows exemplary pictures (taken by Olympus CKX53 cell culture microscope) of fibrillar hydrogel at magnifications of ×4, (FIG. 1A), ×10 (FIG. 1B) and ×20 (FIG. 1C).

FIG. 2 depicts Coomassie-stained SDS-PAGE gel of proteins obtained from the fibrillar hydrogel. Left lane shows kDa scale standard.

FIG. 3 depicts analysis for collagen type I within the fibrillar hydrogel proteins. Proteins separated by SDS-PAGE were transferred to a PVDF membrane, and the membrane proteins were analyzed using a-COL1 antibody. A major band was observed in the expected size (130/139 kDa).

FIG. 4 depicts analysis for collagen type III within the fibrillar hydrogel proteins. Proteins separated by SDS-PAGE were transferred to a PVDF membrane, and the membrane proteins were analyzed using a-COL3A1 antibody. A band was observed in the expected size (140 kDa).

FIG. 5 depicts analysis for collagen type VI within the fibrillar hydrogel proteins. Proteins purified with SDS-PAGE were transferred to a PVDF membrane, and the membrane proteins were analyzed using a-COL16A2 antibody. A band was observed in the expected size (120-160 kDa).

FIG. 6 depicts analysis for collagen type XII within the fibrillar hydrogel proteins. Proteins purified with SDS-PAGE were transferred to a PVDF membrane, and the membrane proteins were analyzed using a-COL12A1 antibody. Several bands were observed, of >185 kDa (expected size: 333 kDa).

FIG. 7 depicts a schematic presentation of the main protein types and protein classes revelated by LC-MS/MS.

FIG. 8 illustrates MS-MS proteomic analysis of the Fibrillar hydrogel-collagen 1 alpha chain 1. (SEQ ID NO: 1). (COL1α1). Gray letters-amino acid residue identified by MS-MS; Black letters—amino acid residue not identified by MS-MS; Shadowed (P)—observed hydroxylation on proline residues; Shadowed (K)—observed hydroxylation on lysine residues; (▾)—hydroxylation on a lysine residue from the literature; (●)—hydroxylation on a proline residue from the literature.

FIG. 9 illustrates MS-MS proteomic analysis of the Fibrillar hydrogel collagen 1 alpha chain 2 (COL1α2) (SEQ ID NO: 2). Gray letters-amino acid residue identified by MS-MS; Black letters-amino acid residue not identified by MS-MS; Shadowed (P)—observed hydroxylation on proline residues; Shadowed (K)—observed hydroxylation on lysine residues; (▾)—hydroxylation on a lysine residue from the literature; (●)—hydroxylation on a proline residue from the literature.

FIG. 10 shows a graph that depicts the relationship between the viscosity (Pas) and shear stress (Pa) of the fibrillar hydrogel produced from bovine fibroblasts. The graph displays the complex, non-linear relationship between viscosity and the shear stress, with viscosity increasing with shear stress up to a yield stress value of 16.2 (Pa), after which the viscosity decreases with further increases in shear stress.

FIG. 11 shows a rheological graph that represents the viscoelastic behavior of the fibrillar hydrogel produced from bovine fibroblasts. The graph displays the Elastic modulus (G′) in Pa, the viscous modulus (G″) Pa, the phase angle (δ) in degrees °, and the Y* parameter in %.

FIG. 12 shows a graph that depicts the relationship between the viscosity (Pa's) and shear stress (Pa) of 3 batches of the fibrillar hydrogel produced from bovine pluripotent stem cells. Batch 1 (∘), Batch 2 (⋄), Batch 3 (□). The graph displays the complex, non-linear relationship between viscosity and the shear stress, with viscosity increasing with shear stress up to a yield stress value of 4 (Pa), after which the viscosity decreases with further increases in shear stress.

FIG. 13 shows a rheological graph that represents the viscoelastic behavior of the fibrillar hydrogel produced from bovine pluripotent stem cells. The graph displays the Elastic modulus (G′) in Pa, the viscous modulus (G″) Pa, the phase angle (δ) in degrees °, and the Y* parameter in %.

### DETAILED DESCRIPTION OF THE INVENTION

The present invention discloses a fibrillar hydrogels comprised of an aqueous solution and a plurality of fibrils, wherein the fibrils are composed of collagen and at least one type of proteoglycan. In exemplary embodiments, the collagen is a non-human animal collagen, particularly type I collagen, the hydrogel further comprises additional ECM proteins, and the hydrogel fibrillar structure is stable at a non-acidic pH.

The fibrillar hydrogels of the invention are advantageous over hitherto known collagen-comprising hydrogels at least in that the hydrogel can be used per se in a variety of applications and manufacturing processes without the need of further priming. The fibrillar hydrogels of the invention is an optimal raw material for sustainable manufacturing of products based on animal-derived materials. According to certain embodiments of the invention, the fibrillar hydrogel may be used in a variety of applications in the textile, pharmaceutic, food, nutraceuticals, and cosmetic industries. In particular, the fibrillar hydrogel of the invention may have a plethora of medical uses, for example for manufacturing wound dressing and/or scaffolds for tissue regeneration.

The collagen-comprising hydrogel of the invention may also be used for textile manufacturing, mainly in manufacturing skin-like or hide-like or leather-like textile, and in the food industry, for the production of cultured meat.

The principles and operation of the present invention may be better understood with reference to the accompanying descriptions. Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Collagen biosynthesis is a complex process comprising intra- and inter-cellular steps. The intracellular steps include gene translation and polypeptide synthesis, particularly synthesis of collagen alpha chains; post-translational modifications, including proline and lysine hydroxylation and glycosylation; and procollagen triple helix formation including proline cis-trans isomerization and stabilization by Heat Shock Protein 47 (HSP47). The soluble procollagen is then secreted to the intercellular space, where it is processed by N- and C-terminal propeptide cleavage to form tropocollagen. Covalent crosslinks are then occurring within and between triple helical collagen molecules to form fibrils, which are assembled to form collagen fibers as part of the ECM.

According to the teachings of the present invention, the majority of the collagen present within the hydrogel is in the form of fibrils. Accordingly, as used herein, the term “collagen” refers to any one of the known collagen types, including collagen types I through XX. The term also encompasses procollagens and any collagenous proteins comprising the motif (Gly-X-Y)n where n is an integer. It encompasses molecules of collagen, trimers of collagen molecules and fibrils of collagen. Collagen types are marked herein by Arabic number (1, 2, 3 etc.) and Roman number (I, II, III etc.) interchangeably.

As used herein, the terms “fibril” or “fibrils” refers to filamentous structure of fibrous structural proteins, mainly of collagen type 1 and at least one type of proteoglycan, including, but not limited to, decorin, biglycan, lumican, prolargin, asporin, fibromodulin, and versican. Each possibility represents a separate embodiment of the present invention. According to certain embodiments, the fibrous structural proteins further comprise at least one of collagen type 3, collagen type 4, collagen type 5, collagen type 6, collagen type 11, collagen type 12, collagen type 14, collagen type 18, fibronectin, fibrillin, vitronectin, elastin, and any combination thereof. Each possibility represents a separate embodiment of the present invention.

The term “proteoglycan” is used herein in its broadest sense as is known in the art. The basic proteoglycan unit consists of a “core protein” with one or more covalently attached glycosaminoglycan (GAG) chains. The point of attachment is a serine residue to which the glycosaminoglycan is joined through a tetra-saccharide bridge. According to some embodiments, the proteoglycan core protein is associated with at least one glycosaminoglycan (GAG) selected from the group consisting of chondroitin sulfate, dermatan sulfate, heparin, heparan sulfate and keratan sulfate. Each possibility represents a separate embodiment of the present invention. According to further embodiments, the fibrillar hydrogel and/or the fibrils comprised therein comprise GAGs which do not form part of the proteoglycan(s). According to certain embodiments, the at least one proteoglycan is selected from the group consisting of: decorin, biglycan, lumican, prolargin, asporin, fibromodulin, versican and any combination thereof. Each possibility represents a separate embodiment of the present invention.

The fibrillar hydrogel of the invention may further contain one or more proteins of the cytoskeleton filament including, but not limited to, actin, vimentin, filamin, and myosin, contributing to the formation of the filamentous structure.

According to certain embodiments, the diameter of the fibrils of the fibrillar hydrogel of the invention is in the range of nanometers to microns, typically from about 100 nm to about 10 μm, and the length can reach millimeters, typically being in the range of from about 100 nm to 10 mm.

As used herein, the term “hydrogel” refers to any dispersion of molecules, fibrils or particles within a liquid in which the solid is the discontinuous phase and the liquid is the continuous phase. According to certain exemplary embodiments, the term “hydrogel” refers to the dispersion of a plurality of fibrils as described hereinabove in an aqueous solution, wherein the hydrogel comprise at least about 30%, 35%, 40%, 45%, 50%, 55%, 60%, 65%, 70%, 75%, 80%, 85%, 90%, 95%, 96%, 97%, 98%, or 99% aqueous solution and at most about 1%, 2%, 3%, 4%, 5%, 10%, 15%, 20%, 25%, 30%, 35%, 40%, 45%, 50%, 55%, 60%, 65% or 70% dry weight. Each possibility represents a separate embodiment of the invention. Accordingly, the hydrogel of the present invention is referred to as “fibrillar hydrogel”. It is to be explicitly understood that an advantage of the fibrillar hydrogel of the present invention resides in that the fibrils are substantially or approximately orderly aligned within the hydrogel, typically in parallel to each other in multiaxial order, contributing to the hydrogel stability and viscoelastic characteristics, and, accordingly, to the elasticity of products produced thereof.

According to some embodiments, at least part of the fibrils are in ordered alignment within the fibrillar hydrogel. According to some embodiments, the aligned fibrils exhibit parallel alignment within the hydrogel. According to some embodiments, the fibrils exhibit an oblique alignment within the hydrogel.

According to some embodiments, at least part of the fibrils exhibit a networked alignment. According to some embodiments, at least part of the network-aligned fibrils are arranged to form a complex interconnected lattice or mesh-like structure within the hydrogel. According to some embodiments, the network-aligned fibrils exhibit a non-linear, interconnected alignment within the hydrogel.

According to some embodiments, the aligned fibrils are biaxially aligned within the hydrogel. According to some embodiments, the aligned fibrils are multiaxially aligned within the hydrogel.

According to some embodiments, between about 30% to 95%, 35% to 90%, 30% to 85%, 30% to 80%, 30% to 75%, 30% to 75%, 30% to 70%, 30% to 65%, 30% to 60%, 30% to 55%, 30% to 50%, 30% to 45% or 30% to 40% of the fibrils are aligned within said hydrogel. Each possibility represents a separate embodiment of the present invention.

According to some embodiments, between about 30% to 35%, 35% to 40%, 40% to 45%, 45% to 50%, 50% to 55%, 55% to 60%, 60% to 65%, 65% to 70%, 70% to 75%, 75% to 80%, 80% to 85% or 90% to 95% of the fibrils are aligned within said hydrogel. Each possibility represents a separate embodiment of the present invention.

According to some embodiments, the fibrils are in alignment from a fibril length of from about 100 nm to about 10 mm. According to some embodiments, the fibrils are in alignment from a fibril length of from about 100 nm to 500 nm, 500 nm to 1 μm, 1 μm to 5 μm, 5 μm to 10 μm, 10 μm to 20 μm, 20 μm to 50 μm, 50 μm to 100 μm, 100 μm to 200 μm, 200 μm to 300 μm, 300 μm to 400 μm, 400 μm to 500 μm, 500 μm to 600 μm, 600 μm to 700 μm, 700 μm to 800 μm, 800 μm to 900 μm, 900 μm to 1 mm, 1 mm to 2 mm, 2 mm to 3 mm, 3 mm to 4 mm, 4 mm to 5 mm, 5 mm to 6 mm, 6 mm to 7 mm, 7 mm to 8 mm, 8 mm to 9 mm or 9 mm to 10 mm. Each possibility represents a separate embodiment of the present invention.

According to some embodiments, the fibrils are in alignment from a fibril length of at least about 100 nm, 500 nm, 1 μm, 5 μm, 10 μm, 20 μm, 50 μm, 200 μm, 300 μm, 400 μm, 500 μm, 600 μm, 700 μm, 800 μm, 900 μm, 1 mm, 2 mm, 3 mm, 4 mm, 5 mm, 6 mm, 7 mm, 8 mm or 9 mm. Each possibility represents a separate embodiment of the present invention.

According to some embodiments, the fibrils are in alignment from a fibril length of no more than about 100 nm, 500 nm, 1 μm, 5 μm, 10 μm, 20 μm, 50 μm, 200 μm, 300 μm, 400 μm, 500 μm, 600 μm, 700 μm, 800 μm, 900 μm, 1 mm, 2 mm, 3 mm, 4 mm, 5 mm, 6 mm, 7 mm, 8 mm or 9 mm. Each possibility represents a separate embodiment of the present invention.

As used herein, the term “plurality” refers to two or more.

As used herein, the term “substantially or approximately aligned” refers to a hydrogel in which the fibrils show more directional uniformity in the matrix as compared to a “random” fibril hydrogel.

As used herein the term “about” refers to +10%.

The terms “comprise”, “comprising”, “include”, “including”, “having” and their conjugates mean “including, but not limited to”.

As used herein, the singular forms “a”, “an” and “the” include plural references unless the context clearly dictates otherwise. For example, the term “a fibril” or “a protein” may include a plurality of fibrils and proteins, including mixtures thereof.

Throughout this application, various embodiments of the invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

According to certain aspects, the present invention provides a fibrillar hydrogel comprising an aqueous solution and a plurality of fibrils, the fibrils comprising collagen and at least one type of proteoglycan, wherein the pH of the hydrogel aqueous solution is at least about 6.00 and wherein at least part of the fibrils are in ordered alignment within said hydrogel.

According to certain aspects, the present invention provides a fibrillar hydrogel comprising an aqueous solution and a plurality of fibrils, the fibrils comprising collagen and at least one type of proteoglycan, wherein at least 40% of the collagen proline and lysine residues of the collagen molecules are hydroxylated and wherein the pH of the hydrogel aqueous solution is at least about 6.00.

According to certain aspects, the present invention provides a fibrillar hydrogel comprising an aqueous solution and a plurality of fibrils, the fibrils comprising collagen and at least one type of proteoglycan, wherein the collagen molecules comprise at least 40% hydroxylated proline and lysine residues out of the total number of proline and lysine residues of said collagen molecules, wherein the pH of the hydrogel aqueous solution is at least about 6.00, and wherein at least part of the fibrils are in ordered alignment within said hydrogel.

According to certain embodiments, the order alignment is a multiaxial alignment.

As used herein, the term “multiaxial alignment” with reference to the fibrils within the hydrogel of the invention refers to an arrangement or orientation of said fibrils along multiple axes or directions, wherein more than one fibril is oriented along a single axes or direction, forming an ordered alignment of the fibrils within said hydrogel.

According to certain embodiments, the aqueous content of the fibrillar hydrogel is at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% out of the total wet weight of the fibrillar hydrogel. Each possibility represents a separate embodiment of the invention.

According to certain exemplary embodiments, the content of the aqueous solution of the fibrillar hydrogel is in the range of from about 90% to about 99%. Each possibility represents a separate embodiment of the invention.

According to certain exemplary embodiments, the content of the aqueous solution of the fibrillar hydrogel is in the range of from about 95% to about 99%. Each possibility represents a separate embodiment of the invention.

According to some embodiments, the aqueous solution is selected from the group consisting of water, a buffer solution and a cell culture medium. According to some embodiments, the buffer is selected from the group consisting of phosphate-buffered saline (PBS), tris-buffered saline (TBS), or 4-(2-hydroxyethyl)-1-piperazineethanesulfonic acid (HEPES) buffer. Each possibility represents a separate embodiment of the invention.

The cell culture medium may be any medium known in the art to be suitable for growing animal cells, particularly mammalian cells. Common cell culture media used in the art include Minimal Essential Medium (MEM), Dulbecco's Modified Eagle's Medium (DMEM), and Roswell Park Memorial Institute-1640 (RPMI-1640). According to certain exemplary embodiments, the aqueous solution is a buffer solution.

According to further certain exemplary embodiments, the aqueous solution is water.

According to certain embodiments, the pH of the aqueous solution is at least 6.1, at least 6.2, at least 6.3, at least 6.4, at least 6.5, at least 6.6, at least 6.7, at least 6.8, at least 6.9, at least 7.0, at least 7.1, at least 7.2, at least 7.3, at least 7.4, at least 7.5, at least 7.6, at least 7.7, at least 7.8, at least 7.9, or at least 8.0 or more. Each possibility represents a separate embodiment of the present invention.

According to certain embodiments, the pH of the aqueous solution is in the range of from about 6.0 to about 8.0, or from about 6.0 to about 7.5, or from about 6.0 to about 7.0.

According to some embodiments, collagen type I is the predominant type of the collagen comprised in the fibril.

According to certain embodiment, collagen type I forms about at least 60% out of the total collagen of the hydrogel. According to some embodiments, collagen type I forms about at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, or more out of the total collagen of the hydrogel. Each possibility represents a separate embodiment of the present invention.

According to certain exemplary embodiments, collagen type I forms from about 60% to about 80% out of the total collagen of the hydrogel.

According to certain embodiments, the fibril comprises collagen type I and at least one additional collagen selected from the group consisting of: Collagen III, collagen IV, collagen VI, collagen XII, collagen V, collagen XI, collagen XIV, collagen II, collagen XI, collagen XVIII and any combination thereof. Each possibility represents a separate embodiment of the present invention.

According to certain embodiments, at least 40%, at least 41%, at least 42%, at least 43%, at least 44%, at least 45%, at least 46%, at least 47%, at least 48%, at least 49%, or at least 50% or more of the Collagen I total number of proline and lysine residues are hydroxylated. Each possibility represents a separate embodiment of the invention.

According to certain embodiments, hydroxylated proline and lysine residues comprise from about 40% to about 60%, or from about 40% to about 55%, or from about 40% to about 50%, of the total number of proline and lysin residues on the Collagen I molecules. Each possibility represents a separate embodiment of the invention.

According certain embodiments, the hydroxylated lysine residues comprise at least 25% out of the total number of lysine residues on the Collagen I molecules. According to certain embodiments, the hydroxylated lysine residues range from about 25% to about 45%, or from about 25% to about 40%, or from 25% to about 35% out of the total lysine residues of the Collagen I molecules. Each possibility represents a separate embodiment of the invention.

According certain embodiments, the hydroxylated proline residues comprise at least 40% out of the total number of proline residues on the Collagen I molecule. According to certain embodiments, the hydroxylated proline residues range from about 40% to about 70%, or from about 40% to about 65%, or from about 40% to about 60%, or from about 40% to about 50% out of the total proline residues of the Collagen I molecule. Each possibility represents a separate embodiment of the invention.

Without wishing to be bound by any theory or mechanism of action, the hydroxylation degree of the collagen proline and lysine residues of the above-described values, contributes to the fibril formation and alignment, and accordingly to the viscoelastic characteristics of the fibrillar hydrogel.

According to certain embodiments, the fibrillar hydrogel dry weight is from about 1% to about 10% out of the total wet weight of said fibrillar hydrogel.

According to certain embodiments, the fibrillar hydrogel dry weight is about 30%, about 35%, about 40%, about 45%, about 50%, or about 55% to about 60%, about 65%, about 70%, out of the total wet weight of said fibrillar hydrogel. Each possibility represents a separate embodiment of the present invention.

According to certain embodiments, the fibrillar hydrogel dry weight is from about 1.5%, about 2.0%, about 2.5%, about 3.0%, about 3.5%, or about 4.0% to about 6%, about 6.5%, about 7.0%, about 7.5%, about 8.0%, or about 8.5% out of the total wet weight of said fibrillar hydrogel. Each possibility represents a separate embodiment of the present invention.

According to certain exemplary embodiments, the fibrillar hydrogel dry weight is from about 2% to about 6% out of the total wet weight of said fibrillar hydrogel.

According to some embodiments, the total amount of collagen is from about 5%, 6%, 7%, 8%, 9%, 10%, 11%, 12%, 13%, 14%, 15%, 16%, 17%, 18%, 19%, or 20% to about 70%, 71%, 72%, 73%, 74%, 75%, 76%, 77%, 78%, 79%, 80%, 81%, 82%, 83%, 84%, 85%, 86%, 87%, 88%, 89%, or 90% w/w out of the total dry weight of the fibrillar hydrogel. Each possibility represents a separate embodiment of the invention.

According to certain embodiments, the total amount of collagen is from about 20% to about 90%, from about 30% to about 90% w/w, from about 40% to about 90%, from about 50% to about 90%, from about 60% to about 90%, from about 70% to about 90% out of the fibrillar hydrogel dry weight.

According to certain exemplary embodiments, the total amount of collagen is from about 70% to 80% w/w out of the fibrillar hydrogel dry weight.

According to some embodiments, the fibrillar hydrogel further comprises hyaluronic acid.

The fibrillar hydrogel of the present invention is produced by animal cells cultured in a culture medium, typically in a suspension. Accordingly, the fibrillar hydrogel typically further comprises additional proteins secreted to the culture medium. Without wishing to be bound by any theory or a mechanism of action, the additional proteins may contribute to the fibril formation and/or to the fibrillar nature of the hydrogel.

According to certain embodiments, the fibrillar hydrogel further comprises at least one protein selected from the group consisting of a glycoprotein, a protein of the cytoskeleton filaments, a cell membrane protein, and any combination thereof. Each possibility represents a separate embodiment of the present invention.

According to certain embodiments, the glycoprotein is selected from the group consisting of fibronectin, serpin H1, Neuroblast differentiation-associated protein AHNAK (desmoyokin), galectin-1, periostin, laminin, peroxidasin, EMILIN, fibrillin, tenascin C, Secreted protein acidic and rich in cysteine (SPARC), and any combination thereof. Each possibility represents a separate embodiment of the present invention.

According to certain embodiments, the fibrillar hydrogel further comprises at least one protein of the cytoskeleton filament. According to certain exemplary embodiments, the protein is selected from the group consisting of: actin, vimentin, myosin, filamin, tropomyosin, lamin, transgelin, tubulin and any combination thereof. Each possibility represents a separate embodiment of the present invention.

According to certain exemplary embodiments the amount of the cytoskeleton filamentous proteins is about 33% w/w out of the total dry weight of the hydrogel. In further exemplary embodiments, the cytoskeleton filamentous proteins are present in the following percentages out of the total dry weight of the hydrogel: actin (10%), Vimentin (6%), Myosin (2.5%), Filamin A (2%), Tropomyosin (1.3%), with Lamin, Elongation factor 1-alpha 1, Transgelin and tubulin comprising the remaining 11.2%.

According to certain embodiments, the amount of the cytoskeleton filament forming proteins out of the total dry weight of the hydrogel, can be reduced by decellularization. According to certain embodiments, the amount of the cytoskeleton filament forming proteins in fibrillar hydrogel undergoing decellularization, can vary within a range of about 1% to 2%, 2% to 3%, 3% to 4%, 4% to 5%, 5% to 6%, 6% to 7%, 7% to 8%, 8% to 9%, 9% to 10%, 10% to 11%, 11% to 12%, 12% to 13%, 13% to 14%, 14% to 15%, 15% to 16%, 16% to 17%, 17% to 18%, 18% to 19% or 19% to 20%. Each possibility represents a separate embodiment of the invention.

According to certain embodiments, the fibrillar hydrogel further comprises at least one cell membranal protein. According to certain exemplary embodiments the cell membranes protein is annexin.

According to certain embodiments, the fibrillar hydrogel is produced by animal cells cultured in a culture medium under conditions enabling collagen synthesis. According to certain exemplary embodiments, the conditions enabling collagen synthesis comprise adding to the cell culture medium at least one collagen synthesis inducing agent.

According to certain embodiments, the agent inducing collagen synthesis is selected from the group consisting of iron, insulin, ascorbic acid or a salt thereof, and a combination of same. Each possibility represents a separate embodiment of the present invention.

According to certain exemplary embodiments, the agent inducing collagen synthesis is ascorbic acid or a salt thereof.

The cells producing collagen can be obtained from any non-human animal having collagen-producing cells as well as from human cells.

According to certain exemplary embodiments, the collagen producing cells are human cells.

According to certain additional or alternative embodiments, the collagen producing cells are of a non-human animal.

According to certain embodiments, the non-human animal is selected from the group consisting of non-human mammals, poultry, aquatic animals, invertebrate and reptiles. Each possibility represents a separate embodiment of the present invention.

According to some embodiments, the non-human animal is known to be used for its skin or hide, including, but not limited to bovine, sheep, horse, crocodile and the like.

According to some embodiments, the collagen-producing cells are of a non-human ungulate.

According to certain embodiments, the ungulate is selected from the group consisting of a bovine, a buffalo, an ovine, an equine, a pig, a giraffe, a camel, a deer, a hippopotamus, and a rhinoceros. Each possibility represents a separate embodiment of the present invention.

According to certain exemplary embodiments, the ungulae is a bovine.

According to further certain exemplary embodiments, the reptile is of the Crocodylidae family.

According to certain embodiments, the cells are stromal cells. According to certain exemplary embodiments, the stromal cells are fibroblast cells.

According to certain additional or alternative embodiments, the cells are collagen-producing cells differentiated from pluripotent stem cells.

According to some embodiments, the fibrillar hydrogel of the present invention has a shape retention at physiological temperatures.

The yield stress values may be the values recorded at temperatures of about 22° C. to about 25° C. from yield stress analysis measurements and values obtained at strain values of 0.04 to 40 Pa.

According to some embodiments, the fibrillar hydrogel of the present invention, having a liquid content of about 90% to about 99%, exhibits a yield stress value in the range of 1 to 50 Pa, 5 to 50 Pa, 10 to 50 Pa, 10 to 40 Pa, 10 to 35 Pa, 10 to 30 Pa, 10 to 25 Pas or 10 to 20 Pa, as determined by a yield stress analysis at a temperature of 25° C. Each possibility represents a separate embodiment of the present invention.

According to certain exemplary embodiments, the yield stress of the fibrillar hydrogel comprising about 90% to about 99% aqueous solution at 25° C., is in the range of from about 10 Pa to about 20 Pa.

The elastic modulus value (G′) of the hydrogel may be the value recorded at temperatures of about 22° C. to about 25° C. from the Oscillatory amplitude sweep analysis. Each possibility represents a separate embodiment of the present invention.

According to some embodiments, the fibrillar hydrogel of the present invention having a liquid content of about 90% to about 99%, has an elastic modulus (G′) (Pa) at temperatures of about 22° C. to about 25° C. of at least about 200 Pa, 210 Pa, 230 Pa, 250 Pa, 270 Pa, 290 Pa, 310 Pa, 330 Pa, or 350 Pa. Each possibility represents a separate embodiment of the present invention.

According to some embodiments, the fibrillar hydrogel of the present invention having a liquid content of about 90% to about 99%, has an elastic modulus (G′) (Pa) at temperatures of about 22° C. to about 25° C. of no more than about 200 Pa, 210 Pa, 230 Pa, 250 Pa, 270 Pa, 290 Pa, 310 Pa, 330 Pa, or 350 Pa. Each possibility represents a separate embodiment of the present invention.

According to some embodiments, the fibrillar hydrogel of the present invention, having a liquid content of about 90% to about 99%, has an elastic modulus (G′) (Pa) at temperatures of about 22° C. to about 25° C. in the range of about 200 Pa to about 350 Pa. Each possibility represents a separate embodiment of the invention. According to certain exemplary embodiments, the fibrillar hydrogel of the present invention having a liquid content of about 90% to about 99%, has an elastic modulus (G′) (Pa) at temperatures of about 22° C. to about 25° C. in the range of about 250 Pa to about 300 Pa. Each possibility represents a separate embodiment of the invention.

The above-described elastic modulus (G′) values are from Oscillatory amplitude sweep analysis.

According to some embodiments, the fibrillar hydrogel of the present invention has a range of elastic moduli (from Oscillatory amplitude sweep analysis) where the minimum and maximum amounts are selected from the embodiments described above.

The phase angle (°) of the hydrogel may be the value recorded at temperatures of about 22° C. to about 25° C. from the Oscillatory amplitude sweep analysis. Each possibility represents a separate embodiment of the present invention.

According to some embodiments, the fibrillar hydrogel of the present invention having a liquid content of about 90% to about 99%, has a phase angle (°) at temperatures of about 22° C. to about 25° C. of at least about 1°, 5°, 10°, 15°, 20°, 25°, 30°, 35°, 40°, 45° or 50°. Each possibility represents a separate embodiment of the present invention.

According to some embodiments, the fibrillar hydrogel of the present invention having a liquid content of about 90% to about 99%, has a phase angle (°) at temperatures of about 22° C. to about 25° C. of no more than about 1°, 5°, 10°, 15°, 20°, 25°, 30°, 35°, 40°, 45° or 50°. Each possibility represents a separate embodiment of the present invention.

According to certain exemplary embodiments, the fibrillar hydrogel of the present invention, having a liquid content of about 90% to about 99%, has a phase angle (°) at temperatures of about 22° C. to about 25° C. in the range of about 5° to about 20°.

The above-described phase angle (°) values are from Oscillatory amplitude sweep analysis.

According to some embodiments, the fibrillar hydrogel of the present invention having a liquid content of about 90% to about 99% at a temperature in a range of from about 22° C. to about 26° C. is characterized by at least one of a yield stress of from about 1 Pa to about 50 Pa, an elastic modulus (G′) of from about 200 Pa to about 350 Pa, a phase angle (°) of from about 1° to about 50°, and any combinations thereof. Each possibility represents a separate embodiment of the present invention.

According to certain embodiments, the fibrillar hydrogel of the present invention having a liquid content of about 90% to about 99% at a temperature in a range of from about 22° C. to about 26° C. is characterized by at least one of a yield stress of about 16 Pa, an elastic modulus (G′) in the range of about 250 Pa to about 300 Pa, a phase angle (°) of about 15°, and any combinations thereof. Each possibility represents a separate embodiment of the present invention.

According to certain exemplary embodiments, the fibrillar hydrogel of the present invention having a liquid content of about 90% to about 99% at a temperature in a range of from about 22° C. to about 26° C., has a yield stress of about 16 Pa, an elastic modulus (G′) in the range of about 250 Pa to about 300 Pa, and a phase angle (°) of about 15°.

According to some embodiments, the rheological properties of the fibrillar hydrogel of the present invention remain substantially the same after at least one cycle of deformation and reassembly, such as two cycles of deformation and reassembly. The rheological property may be one or more properties selected from the group consisting of yield stress, elastic modulus, and phase angle.

According to some embodiments, the fibrillar hydrogel of the present invention demonstrates a high elastic modulus (G′) compared to its viscous modulus (G″), indicating a more solid-like behavior while still maintaining some liquid-like characteristics.

According to some embodiments, the fibrillar hydrogel is substantially free of pyrogens. According to some embodiments, the fibrillar hydrogel is substantially free of endotoxins. According to some embodiments, the fibrillar hydrogel of the present invention is substantively non-cytotoxic. According to some embodiments, the fibrillar hydrogel of the present invention is substantially free of residual monomers.

The fibrillar hydrogel of the present invention is suitable for a variety of uses. According to certain embodiments, the fibrillar hydrogel of the invention is used as a biomaterial. According to certain embodiments, the biomaterial is for the production of textiles, particularly skin- or hide- or leather-like textiles. The fibrillar hydrogel is suitable for use in a variety of textile-production methods, as are currently known and as will be known in the art. According to certain embodiments, the biomaterial is for medical use, for example for the construction of artificial organs or prostheses or for replacement of bone or tissue. The fibrillar hydrogel of the present invention is further suitable for use in the food industry. In certain exemplary embodiments, the fibrillar hydrogel of the invention is used in the production of cultured meat. The fibrillar hydrogel may be used in its hydrogel form or may be bio-fabricated as a scaffold for cell growth.

According to some embodiments, the fibrillar hydrogel of the present invention is suitable for use in the formulation of nutraceuticals, dietary supplements, and food additives. Each possibility represents a separate embodiment of the present invention.

According to some embodiments, the fibrillar hydrogel of the present invention is suitable for use as a fat alternative in low-calorie or low-fat food products, providing a similar mouthfeel and texture without the added calories or fat content.

According to some embodiments, the fibrillar hydrogel of the present invention is suitable for use as a thickening or gelling agent in various food products, such as sauces, soups, and dressings.

According to some embodiments, the fibrillar hydrogel of the present invention is suitable for use as a binding agent in meat, plant-based protein products, or cultured meat products improving texture and cohesion.

According to some embodiments, the fibrillar hydrogel of the present invention is suitable for use as a moisture-retaining agent in baked goods, enhancing their texture and shelf life. According to some embodiments, the fibrillar hydrogel of the present invention is suitable for use as a texture modifier in gluten-free food products, improving the overall mouthfeel and consistency.

According to some embodiments, the fibrillar hydrogel of the present invention is suitable for use as a moisture-absorbing agent in the production of crispy food products, such as dried cultured meat products, maintaining their desired texture.

According to some embodiments, the fibrillar hydrogel of the present invention is suitable for medical uses.

According to some embodiments, the fibrillar hydrogel is suitable for use as a wound dressing to promote healing and tissue regeneration. According to these embodiments, the fibrillar hydrogel may further comprise at least one active agent, selected from the group consisting of an antimicrobial agent, a debriding agent, a blood-clotting promoting agent, a cell growth and/or tissue regeneration factor, an anti-inflammatory agent, a pain reliver and any combination thereof. Each possibility represents a separate embodiment of the present invention.

According to some embodiments, the fibrillar hydrogel is suitable for use as a joint filler to provide support, cushioning, and promote cartilage regeneration in damaged joints. According to some embodiments, the joint is knee joint.

According to some embodiments, the fibrillar hydrogel is suitable for use as a surgical mesh for tissue reinforcement.

According to some embodiments, the fibrillar hydrogel is suitable for use as a controlled-release drug delivery system, releasing therapeutic agents over time to targeted areas.

According to some embodiments, the fibrillar hydrogel is suitable for use as a scaffold for the regeneration of blood vessels.

According to some embodiments, the fibrillar hydrogel is suitable for use as a supportive scaffold for skin grafts.

According to some embodiments, the fibrillar hydrogel is suitable for use as an injectable filler for soft tissue augmentation, such as in facial rejuvenation procedures or breast augmentation.

According to some embodiments, the fibrillar hydrogel is suitable for use as a cell culture scaffold, providing a supportive environment for the growth and differentiation of various cell types.

According to some embodiments, the fibrillar hydrogel is suitable for use as in tissue engineering applications to create custom tissue constructs for implantation, such as in reconstructive surgeries.

According to some embodiments, the fibrillar hydrogel is used in guided tissue regeneration. Collagen-based membranes can be used in periodontal and implant therapy to promote the growth of specific types of cells.

According to some embodiments, the fibrillar hydrogel of the present invention is suitable for cosmetic uses. According to some embodiments, the fibrillar hydrogel is used as a skin filler. Fillers that contain collagen can be used cosmetically to remove lines and wrinkles from the face and can also improve scars.

According to some embodiments, the fibrillar hydrogel of the present invention is suitable for use in non-surgical nose reshaping.

According to some embodiments, the fibrillar hydrogel of the present invention is suitable for use in eyebrow enhancement, creating a natural-looking, semi-permanent solution for fuller brows.

According to some embodiments, the fibrillar hydrogel of the present invention is suitable for use in skin tightening treatments.

According to some embodiments, the fibrillar hydrogel of the present invention is suitable for use in cosmetic augmentation including breast and buttocks.

According to some embodiments, the fibrillar hydrogel of the present invention is suitable for use in the treatment of stretch marks. According to some embodiments, the fibrillar hydrogel of the present invention is suitable for use in the treatment of dark circles under the eyes.

An additional advantage of the fibrillar hydrogel of the invention is that it may be used in the above-described applications without the need of any further processes.

According to some embodiments, the fibrillar hydrogel is used for the production of synthetic leather. According to some embodiments, the fibrillar hydrogel is used for the production of leather-mimetic textiles.

According to some embodiments, the fibrillar hydrogel is used in the production of woven textiles.

According to some embodiments, the fibrillar hydrogel is used for the creation of non-woven textiles.

According to some embodiments, the fibrillar hydrogel is used in the production of textiles for medical applications, such as wound dressings or sutures.

According to some embodiments, the fibrillar hydrogel is used in the production of environmentally friendly textiles.

According to additional certain aspects, the present invention provides a textile comprising at least 10% of the fibrillar hydrogel of the invention. According to certain embodiments, the textile has skin/hide/leather-like characteristics. According to certain embodiments, the textile comprises from about 10%, 11%, 12%, 13%, 14%, 15%, 16%, 17%, 18%, 19%, or 20% to about 30%, 35%, 40%, 45%, 50% or 60% of the fibrillar hydrogel. Each possibility represents a separate embodiment of the present invention.

According to yet additional certain aspects, the present invention provides a food product comprising at least 0.5% of the fibrillar hydrogel of the invention. According to certain embodiments, the food product comprises from about 0.6%, 0.7%, 0.8%, 0.9%, 1%, 1.1%, 1.3%, 1.3%, 1.4%, 1.5%, 1.6%, 1.7%, 1.8%, 1.9%, or 2% to about 5%, 5.5%, 6%, 6.5%. 7%, 7.5%, 8%, 8.5%, 9%, 9.5% or 10% of the fibrillar hydrogel. According to certain embodiments, the food product is a cell cultured meat product. Each possibility represents a separate embodiment of the present invention.

According to further certain aspects, the present invention provides a medical material comprising at least 0.1% of the fibrillar hydrogel of the invention. According to certain embodiments, the medical material comprises from about 0.1%, 0.2%, 0.3%, 0.4%, 0.5%, 0.6%, 0.7%, 0.8%, or 0.9%, to about 1%, 1%, 1.1%, 1.3%, 1.3%, 1.4%, 1.5%, 1.6%, 1.7%, 1.8%, 1.9%, or 2% of the fibrillar hydrogel. Each possibility represents a separate embodiment of the present invention.

According to yet further certain aspects, the present invention provides a cosmetic product comprising at least 0.01% of the fibrillar hydrogel of the invention. According to certain embodiments, the cosmetic comprises from about 0.01%, 0.02%, 0.03%, 0.04%, 0.05%, 0.06%, 0.07%, 0.0%, 0.09%, or 0.1% to about 1%, 1%, 1.1%, 1.3%, 1.3%, 1.4%, 1.5%, 1.6%, 1.7%, 1.8%, 1.9%, or 2% of the fibrillar hydrogel. Each possibility represents a separate embodiment of the present invention.

According to yet additional certain aspects, the present invention provides a nutraceutical product comprising at least 1% of the fibrillar hydrogel of the invention. According to certain embodiments, the nutraceutical product comprises from about 1%, 2%, 3%, 3%, 4%, or 5% to about 15%, 16%, 17%, 18%, 19%, or 20% of the fibrillar hydrogel. Each possibility represents a separate embodiment of the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

### Example 1: Cell Culturing and Harvesting of Fibrillar Hydrogel

### 1.1 Fibrillar Hydrogel Production Using Bovine Fibroblast

Bovine embryonic fibroblast cells were expanded in standard medium (DMEM+10% Fetal Bovine Serum). 24 hours after seeding, the medium was changed to basal medium supplemented with ascorbic acid (150 mg/L) to increase the induction of collagen. Following 6 days of induction, the medium was aspirated and the cells were washed with DPBS without calcium and magnesium. Pre-warmed recombinant trypsin EDTA was added, and the cells were incubated at 38.5° C. for 5 minutes until thin fibrillar hydrogel appeared. To dilute the trypsin, DMEM in an equal volume of trypsin was added and the fibrillar hydrogel was collected to a sterile tube that was stored at 4° C.

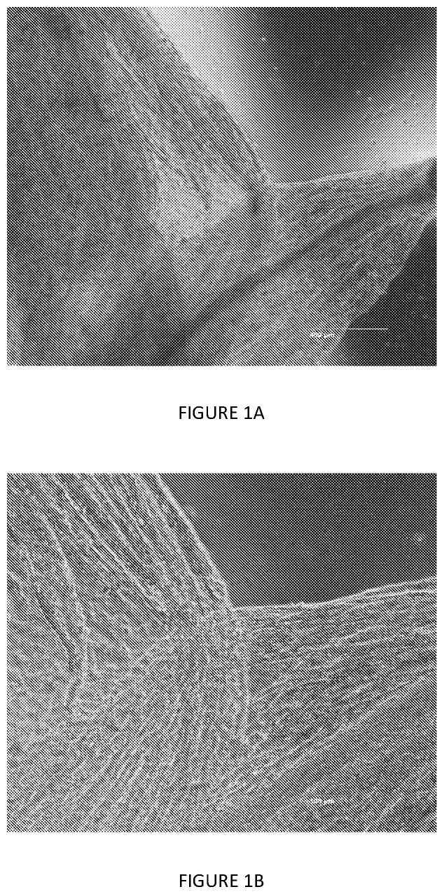

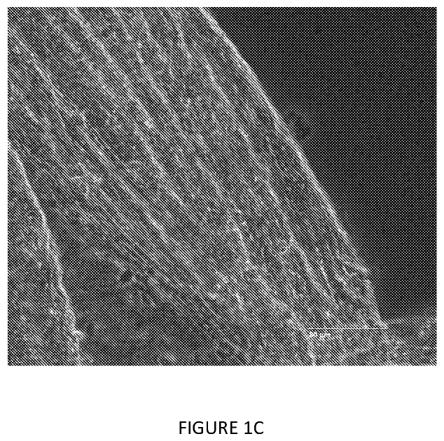

Exemplary optical-microscope pictures (Olympus CKX53 cell culture microscope) showing the organized structure of the fibrils within the hydrogel are presented in FIG. 1A-C.

### 1.2 Fibrillar Hydrogel Production Using Bovine Pluripotent Stem Cells (PSCs)

Bovine pluripotent stem cells (PSCs) were grown in 3D culture. The PSCs were dissociated and resuspended in a serum-free growth medium containing Activin A (20 ng/ml) and CHIR-99021 5 (10 μM) for a period of 4 days. A Rho-associated kinase (ROCK) Inhibitor (10 μM) was introduced during the first day of growth. Following the 4-day incubation, the PSCs were dissociated and seeded as single cells in standard medium Dulbecco's Modified Eagle's Medium (DMEM) and 10% Fetal Bovine Serum (FBS). The medium was changed every 48 hours for 7 days. Next, ascorbic acid (150 mg\L) was added to the standard medium to increase the induction of collagen synthesis. After an additional 7-day incubation period, the medium was aspirated, and the cells were washed with Dulbecco's Phosphate Buffered Saline (DPBS) devoid of calcium and magnesium. Subsequently, pre-warmed recombinant trypsin-ethylenediaminetetraacetic acid (EDTA) solution was added to the cells, and the culture was incubated at 38.5° C. for 5 minutes until a thin fibrillar hydrogel was observed. The trypsin activity was neutralized by the addition of an equal volume of DMEM. The resulting fibrillar hydrogel was washed three times with DPBS, followed by storage at 4° C.

### Example 2: SDS-PAGE Analysis of Harvested Fibrillar Hydrogel

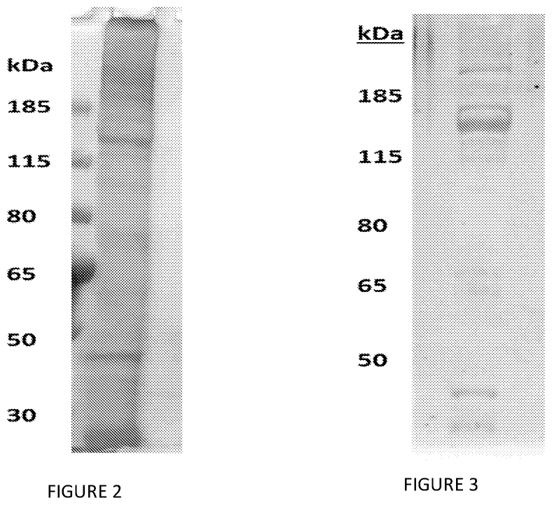

The fibrillar hydrogel (produced from bovine fibroblasts as described in Example 1 hereinabove) was resuspended in 8M urea. The soluble fraction was mixed with reducing sample buffer, boiled at 95° C. for 5 min and loaded on an 8% SDS-PAGE. After running, the gel was stained using Coomassie dye (FIG. 2).

### Example 3: Anti-COL1 Analysis of Harvested Fibrillar Hydrogel

The fibrillar hydrogel (produced from bovine fibroblasts as described in Example 1 hereinabove) was resuspended in 8M urea. The soluble fraction was mixed with reducing sample buffer, boiled at 95° C. for 5 min and loaded on an 8% SDS-PAGE. Then, the proteins were transferred to a PVDF membrane, and the membrane was analyzed for the presence of collagen type I using a-COL1 antibody. A major band was observed in the expected size (130/139 kDa, FIG. 3).

### Example 4: Anti-COL3 Analysis of Harvested Fibrillar Hydrogel

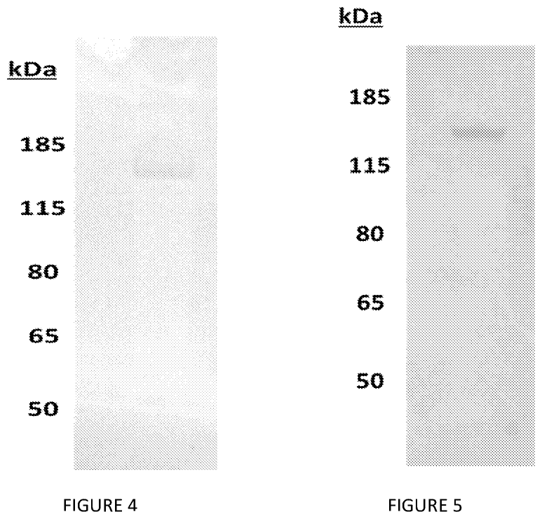

The fibrillar hydrogel (produced from bovine fibroblasts as described in Example 1 hereinabove) was resuspended in 8M urea. The soluble fraction was mixed with reducing sample buffer, boiled at 95° C. for 5 min and loaded on an 8% SDS-PAGE. Then, the proteins were transferred to a PVDF membrane, and the membrane proteins were analyzed for the presence of collagen type III using a-COL3A1 antibody. A band was observed in the expected size (140 kDa, FIG. 4).

### Example 5: Anti-COL6 Analysis of Harvested Fibrillar Hydrogel

The fibrillar hydrogel (produced from bovine fibroblasts as described in Example 1 hereinabove) was resuspended in 8M urea. The soluble fraction was mixed with reducing sample buffer, boiled at 95° C. for 5 min and loaded on an 8% SDS-PAGE. Then, the proteins were transferred to a PVDF membrane, and the membrane proteins were analyzed for the presence of collagen type VI using a-COL6A2 antibody. A band was observed in the expected size (120-160 kDa, FIG. 5)

### Example 6: Anti-COL12 Analysis of Harvested Fibrillar Hydrogel

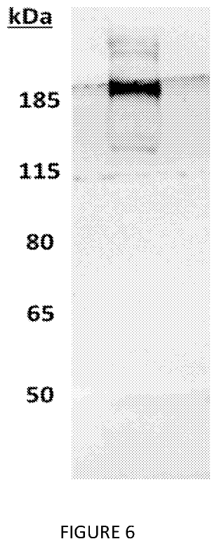

The fibrillar hydrogel (produced from bovine fibroblasts as described in Example 1 hereinabove) was resuspended in 8M urea. The soluble fraction was mixed with reducing sample buffer, boiled at 95° C. for 5 min and loaded on an 8% SDS-PAGE. Then, the proteins were transferred to a PVDF membrane, and the membrane proteins were analyzed for the presence of collagen type XII using a-COL12A1 antibody. Several bands were observed, of >185 kDa (expected size: 333 kDa, FIG. 6).

### Example 7: Analysis of the Fibrillar Hydrogel Protein Content

To analyze the protein contents of the fibrillar hydrogel produced as described in Example 1 (from bovine-derived fibroblasts and from bovine-derived PSC differentiated to collagen-producing cells), a liquid chromatography-tandem mass spectrometry (LC-MS/MS) was applied on each preparation. Samples were digested by trypsin, analyzed by LC-MS/MS on Q-Exactive plus (Thermo) and identified by Discoverer software against the bovine proteome from the Uniprot database, and a decoy database (in order to determine the false discovery rate). All the identified peptides were filtered with high confidence, and the identified proteins with a minimum of 2 peptides.

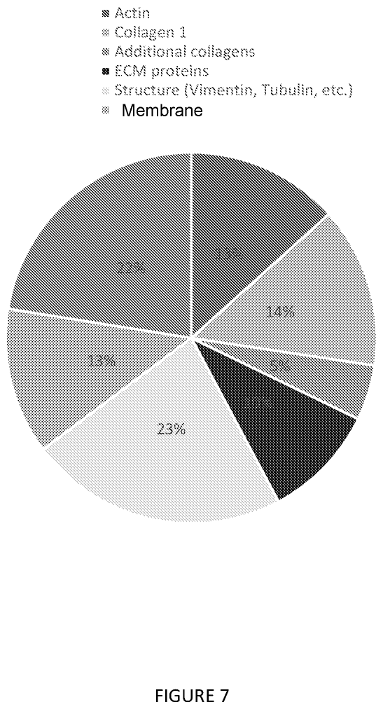

For the LC-MS/MS, High confidence peptides have passed the 1% FDR threshold. (*FDR=false discovery rate, is the estimated fraction of false positives in a list of peptides). A protein identified with a single unique peptide could not be considered as a certain identification. Major proteins and protein classes are schematically presented in FIG. 7.

### Example 8: MS-MS Proteomic Analysis of Collagen 1 Alpha Chain 1 (COL1A1)

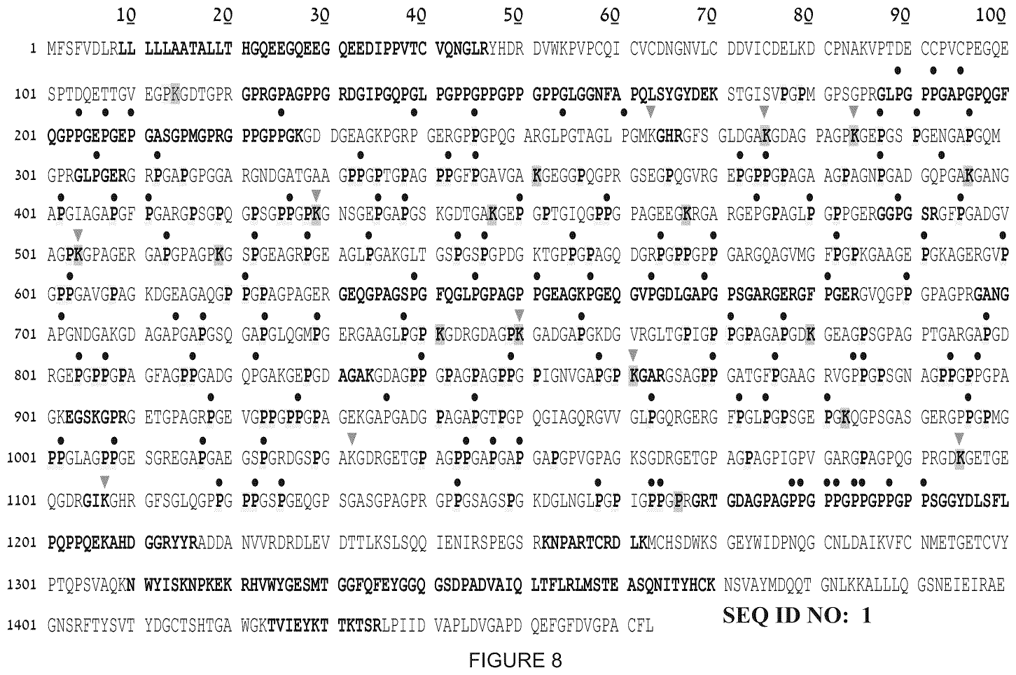

Samples of the fibrillar hydrogel (produced as described in Example 1 hereinabove) were analyzed by liquid chromatography-tandem mass spectrometry. Preparing the sample for MS-MS ((LC-MS)/MS) included trypsin digestion. Sequences and post-translational modifications (PTMs) were then compared to those of bovine COL1A1 (AAI05185.1) from NCBI protein database (FIG. 8).

### Example 9: MS-MS Proteomic Analysis of the Collagen 1 Alpha Chain 2 (COL1A2)

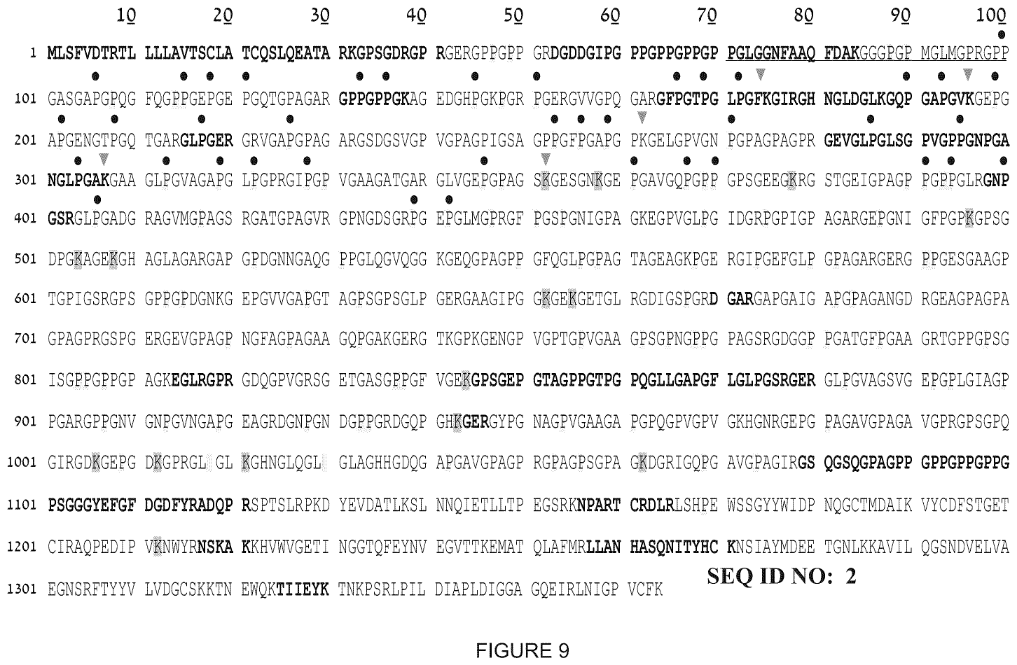

As described in Example 8 hereinabove, samples of the fibrillar hydrogel were analyzed by liquid chromatography-tandem mass spectrometry ((LC-MS)/MS) following trypsin digestion. Sequences and PTMs were then compared to those of bovine COL1A2 (NP_776945.1) from NCBI protein database (FIG. 9).

### Example 10: Yield Stress Analysis of the Fibrillar Hydrogel Produced from Bovine Fibroblasts and PSCs

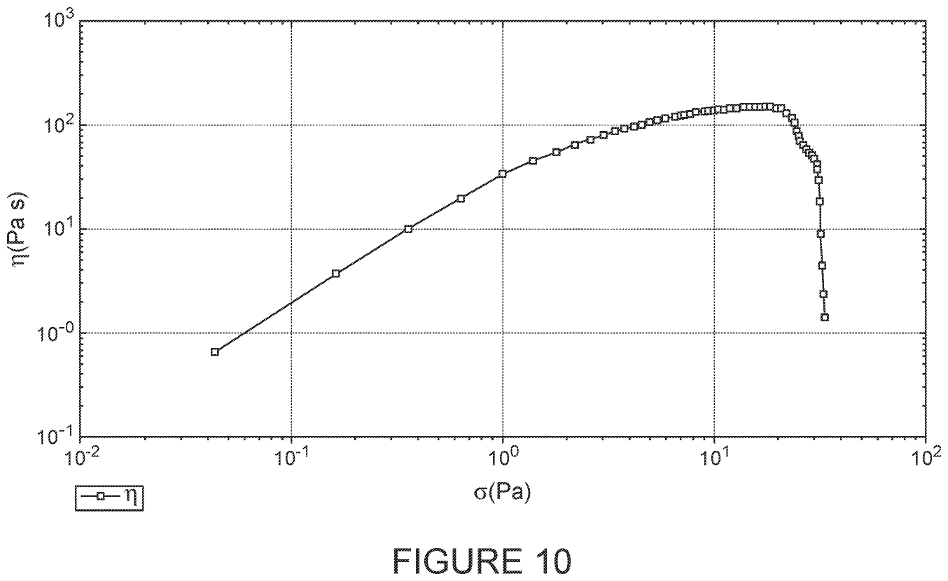

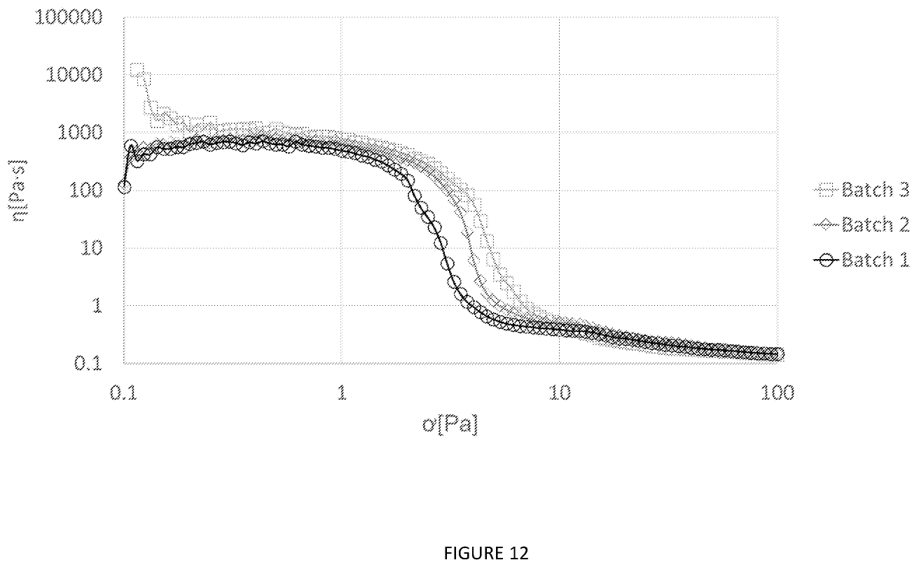

The shear stress of a sample of the fibrillar hydrogel (produced as described in Section 1.1, Example 1 hereinabove) was analyzed using the +Pro Kinexus Netzsch rheometer at a temperature of 25° C. (FIG. 10). Fibrillar hydrogel samples (produced as described in Section 1.2, Example 1) (Batch 1, Batch 2, and Batch 3) were analyzed using the MCR 102e rheometer (Anton Paar) at a temperature of 25° C. (FIG. 12).

Each sample was placed between two parallel plates, with one fixed and the other rotating in one direction (shear flow) or oscillating back and forth (oscillatory shear). The rheological behavior of the hydrogel was studied over a range of stress conditions, and yield stress values of 16.2 Pa and 4 Pa was observed for the fibrillar hydrogel samples produced from bovine fibroblasts and PSCs, respectively.

### Example 11: Oscillatory Amplitude Sweep Analysis of the Fibrillar Hydrogel Produced from Bovine Fibroblasts and PSCs

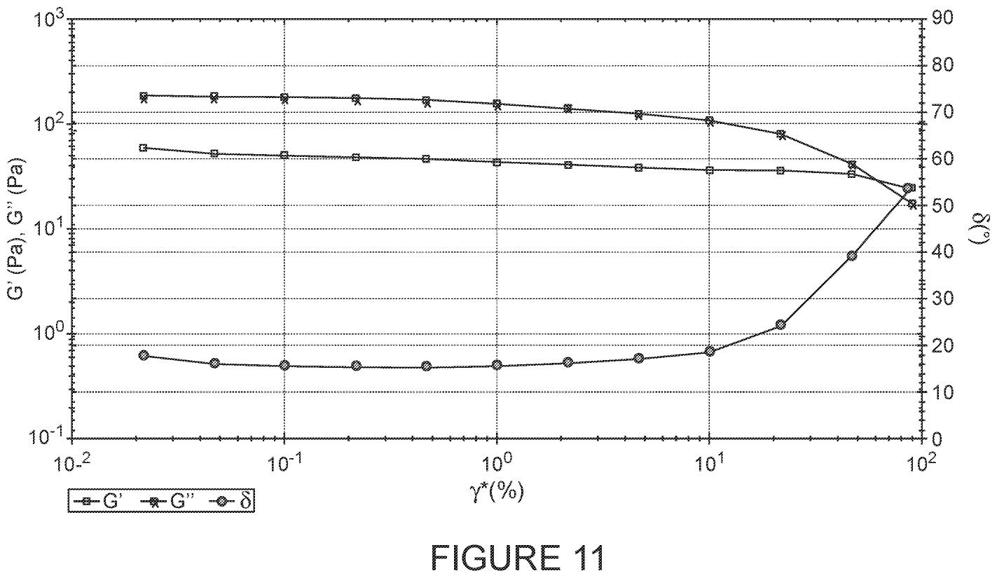

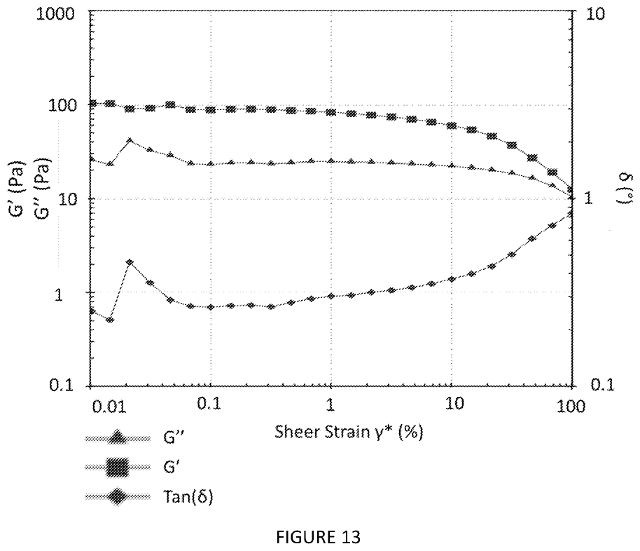

The viscoelastic behavior of a sample of the fibrillar hydrogel (produced as described in Section 1.1, Example 1 hereinabove) was analyzed using the +Pro Kinexus Netzsch rheometer (FIG. 11) with an oscillatory amplitude sweep protocol. A fibrillar hydrogel sample (produced as described in Section 1.2, Example 1) was analyzed using the Modular Compact Rheometer (MCR) 102e (Anton Paar) (FIG. 13) with an oscillatory amplitude sweep protocol.

Each sample was placed between two plates, with the upper plate oscillating back and forth in both directions at a constant frequency but with an increased amplitude at each cycle, with the shear stress being increased accordingly. The experiment was carried out at 25° C.

The results of the oscillatory amplitude sweep analysis showed that the fibrillar hydrogel samples exhibited a viscoelastic response with both elastic (G′) and viscous (G″) components. The Elastic modulus (G′) in Pa (blue) and viscous modulus (G″) (red) were plotted against the Y* parameter. The hydrogel exhibited predominantly solid-like behavior at low Y* values, as indicated by a higher G′ than G″ value, and a phase angle (δ) of about 15° and 16.8° for the fibrillar hydrogel samples produced from bovine fibroblasts and PSCs, respectively. This suggests that the fibrillar hydrogel is a semi-solid material that behaves more like a solid.

### Example 12: PH Analysis of the Fibrillar Hydrogel Produced Using Bovine Pluripotent Stem Cells (PSCs)

The pH of the fibrillar hydrogel (produced as described in Example 1 hereinabove) was analyzed using a pH meter (Knick Portavo 907 Multi). The fibrillar hydrogel sample was placed in a 50 mL tube. The pH meter sensor was then inserted into the sample, measuring a pH of 7.15.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A fibrillar hydrogel comprising an aqueous solution and a plurality of fibrils, the fibrils comprising collagen and at least one type of proteoglycan, wherein the pH of the hydrogel aqueous solution is at least about 6.00 and wherein at least part of the fibrils are in ordered alignment within said hydrogel.

2. The fibrillar hydrogel of claim 1, wherein the ordered alignment is selected from the group consisting of biaxial alignment and multiaxial alignment.

3. The fibrillar hydrogel of claim 1, wherein the collagen molecules comprise at least 40% hydroxylated proline and lysine residues out of the total number of proline and lysine residues of said collagen molecules.

4. (canceled)

5. The fibrillar hydrogel of claim 1, wherein the aqueous content of said fibrillar hydrogel is at least 30% w/w out of the total weight of said fibrillar hydrogel.

6. (canceled)

7. The fibrillar hydrogel of claim 1, wherein the fibril comprises at least one collagen type selected from the group consisting of collagen I, collagen VI, collagen XII, collagen V, collagen XIV, collagen II, collagen XI, collagen III and any combination thereof, and wherein collagen I forms at least 60% out of the total collagen content of said hydrogel.

8-10. (canceled)

11. The fibrillar hydrogel of claim 1, wherein the at least one type of proteoglycan is selected from the group consisting of: decorin, biglycan, lumican, prolargin, asporin, fibromodulin, versican, and any combination thereof.

12. (canceled)

13. The fibrillar hydrogel of claim 1, wherein said fibrillar hydrogel comprises from about 20% to about 90% w/w total collagen out of the total dry weight of said hydrogel.

14. The fibrillar hydrogel of claim 1, wherein said fibrillar hydrogel further comprises at least one additional protein selected from the group consisting of a glycoprotein, a protein of the cytoskeleton filaments, a cell membrane protein, and any combination thereof.

15-17. (canceled)

18. The fibrillar hydrogel of claim 1, wherein said fibrillar hydrogel further comprises hyaluronic acid.

19. The fibrillar hydrogel of claim 1, wherein said fibrillar hydrogel, comprising an aqueous solution content of from about 90% to about 99%, at a temperature in a range of from about 22° C. to about 26° C. is characterized by a yield stress of from about 1 Pa to about 50 Pa.

20. (canceled)

21. The fibrillar hydrogel of claim 1, wherein said fibrillar hydrogel, comprising an aqueous solution content of from about 90% to about 99%, at a temperature in a range of from about 22° C. to about 26° C. is characterized by a phase angle of from about 1° to about 50°.

22. (canceled)

23. The fibrillar hydrogel of claim 1, wherein said fibrillar hydrogel is produced by animal cells capable of producing collagen, cultured in a culture medium under conditions enabling collagen synthesis.

24-28. (canceled)

29. The fibrillar hydrogel of claim 23, wherein said fibrillar hydrogel is substantially free of pyrogens.

30-41. (canceled)

42. A food product comprising at least 0.5% of the fibrillar hydrogel of claim 1.

43. (canceled)

44. The food product of claim 42, wherein said food product is cultured meat.

45. A cosmetic product comprising at least 0.01% of the fibrillar hydrogel of claim 1.

46. (canceled)

47. A medical product comprising at least 0.1% of the fibrillar hydrogel of claim 1.

48-50. (canceled)

51. The fibrillar hydrogel of claim 23, wherein said fibrillar hydrogel is substantially free of the animal cells.

52. A nutraceutical product comprising at least 1% of the fibrillar hydrogel of claim 1.

53. A textile comprising at least 10% of the fibrillar hydrogel of claim 1.


SEQUENCE LISTING (2 sequences)

SEQ ID NO: 1 - protein, 1462 aa, Bos taurus
MFSFVDLRLLLLLAATALLTHGQEEGQEEGQEEDIPPVTCVQNGLRYHDRDVWKPVPCQICVCDNGNVLCDDVICDELKDCPNAKVPTDECCPVCPEGQESPTDQETTGVEGPKGDTGPRGPRGPAGPPGRDGIPGQPGLPGPPGPPGPPGPPGLGGNFAPQLSYGYDEKSTGISVPGPMGPSGPRGLPGPPGAPGPQGFQGPPGEPGEPGASGPMGPRGPPGPPGKGDDGEAGKPGRPGERGPPGPQGARGLPGTAGLPGMKGHRGFSGLDGAKGDAGPAGPKGEPGSPGENGAPGQMGPRGLPGERGRPGAPGPGGARGNDGATGAAGPPGPTGPAGPPGFPGAVGAKGEGGPQGPRGSEGPQGVRGEPGPPGPAGAAGPAGNPGADGQPGAKGANGAPGIAGAPGFPGARGPSGPQGPSGPPGPKGNSGEPGAPGSKGDTGAKGEPGPTGIQGPPGPAGEEGKRGARGEPGPAGLPGPPGERGGPGSRGFPGADGVAGPKGPAGERGAPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGAPGKDGVRGLTGPIGPPGPAGAPGDKGEAGPSGPAGPTGARGAPGDRGEPGPPGPAGFAGPPGADGQPGAKGEPGDAGAKGDAGPPGPAGPAGPPGPIGNVGAPGPKGARGSAGPPGATGFPGAAGRVGPPGPSGNAGPPGPPGPAGKEGSKGPRGETGPAGRPGEVGPPGPPGPAGEKGAPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPIGPVGARGPAGPQGPRGDKGETGEQGDRGIKGHRGFSGLQGPPGPPGSPGEQGPSGASGPAGPRGPPGSAGSPGKDGLNGLPGPIGPPGPRGRTGDAGPAGPPGPPGPPGPPGPPSGGYDLSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKEKRHVWYGESMTGGFQFEYGGQGSDPADVAIQLTFLRLMSTEASQNITYHCKNSVAYMDQQTGNLKKALLLQGSNEIEIRAEGNSRFTYSVTYDGCTSHTGAWGKTVIEYKTTKTSRLPIIDVAPLDVGAPDQEFGFDVGPACFL

SEQ ID NO: 2 - protein, 1364 aa, Bos taurus
MLSFVDTRTLLLLAVTSCLATCQSLQEATARKGPSGDRGPRGERGPPGPPGRDGDDGIPGPPGPPGPPGPPGLGGNFAAQFDAKGGGPGPMGLMGPRGPPGASGAPGPQGFQGPPGEPGEPGQTGPAGARGPPGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGELGPVGNPGPAGPAGPRGEVGLPGLSGPVGPPGNPGANGLPGAKGAAGLPGVAGAPGLPGPRGIPGPVGAAGATGARGLVGEPGPAGSKGESGNKGEPGAVGQPGPPGPSGEEGKRGSTGEIGPAGPPGPPGLRGNPGSRGLPGADGRAGVMGPAGSRGATGPAGVRGPNGDSGRPGEPGLMGPRGFPGSPGNIGPAGKEGPVGLPGIDGRPGPIGPAGARGEPGNIGFPGPKGPSGDPGKAGEKGHAGLAGARGAPGPDGNNGAQGPPGLQGVQGGKGEQGPAGPPGFQGLPGPAGTAGEAGKPGERGIPGEFGLPGPAGARGERGPPGESGAAGPTGPIGSRGPSGPPGPDGNKGEPGVVGAPGTAGPSGPSGLPGERGAAGIPGGKGEKGETGLRGDIGSPGRDGARGAPGAIGAPGPAGANGDRGEAGPAGPAGPAGPRGSPGERGEVGPAGPNGFAGPAGAAGQPGAKGERGTKGPKGENGPVGPTGPVGAAGPSGPNGPPGPAGSRGDGGPPGATGFPGAAGRTGPPGPSGISGPPGPPGPAGKEGLRGPRGDQGPVGRSGETGASGPPGFVGEKGPSGEPGTAGPPGTPGPQGLLGAPGFLGLPGSRGERGLPGVAGSVGEPGPLGIAGPPGARGPPGNVGNPGVNGAPGEAGRDGNPGNDGPPGRDGQPGHKGERGYPGNAGPVGAAGAPGPQGPVGPVGKHGNRGEPGPAGAVGPAGAVGPRGPSGPQGIRGDKGEPGDKGPRGLPGLKGHNGLQGLPGLAGHHGDQGAPGAVGPAGPRGPAGPSGPAGKDGRIGQPGAVGPAGIRGSQGSQGPAGPPGPPGPPGPPGPSGGGYEFGFDGDFYRADQPRSPTSLRPKDYEVDATLKSLNNQIETLLTPEGSRKNPARTCRDLRLSHPEWSSGYYWIDPNQGCTMDAIKVYCDFSTGETCIRAQPEDIPVKNWYRNSKAKKHVWVGETINGGTQFEYNVEGVTTKEMATQLAFMRLLANHASQNITYHCKNSIAYMDEETGNLKKAVILQGSNDVELVAEGNSRFTYYVLVDGCSKKTNEWQKTIIEYKTNKPSRLPILDIAPLDIGGAGQEIRLNIGPVCFK